(19) United States
(12) Patent Application Publication
(10) Pub. No.: US 2024/0122760 A1
(43) Pub. Date: Apr. 18, 2024

(54) METHOD FOR ADAPTING TREATMENT COORDINATES FOR A TREATMENT WITH AN OPHTHALMOLOGICAL LASER

(71) Applicant: SCHWIND EYE-TECH-SOLUTIONS GMBH, Kleinostheim (DE)

(72) Inventor: Samuel ARBA MOSQUERA, Aschaffenburg (DE)

(21) Appl. No.: 18/474,737

(22) Filed: Sep. 26, 2023

(30) Foreign Application Priority Data
Oct. 14, 2022 (DE) 10 2022 126 857.8

(51) Int. Cl. A61F 9/009; A61F 9/008

(52) U.S. Cl. CPC A61F 9/009; A61F 9/00802; A61F 9/00825; A61F 2009/00855; A61F 2009/00872; A61F 2009/00878; A61F 2009/00897

(57) ABSTRACT

A method is disclosed for adapting treatment coordinates for a treatment of an eye with an ophthalmological laser of a treatment apparatus. The treatment apparatus includes a contact element for fixing the eye. The method includes acquiring at least a first image of the eye, before the eye is fixed by the contact element, and determining treatment coordinates of the eye by means of the first image, determining orientation points of the eye and the position thereof in the first image; acquiring a second image of the eye, after the eye has been fixed by the contact element, wherein the position of the respective orientation points is determined in the second image. The method also includes determining a transformation matrix based on the respectively determined positions of related orientation points in the first and the second image, and adapting (S18) the treatment coordinates by the determined transformation matrix.

### FIELD

The present invention relates to a method for adapting treatment coordinates for a treatment of an eye with an ophthalmological laser of a treatment apparatus. In addition, the invention relates to a control device for performing the method, to a treatment apparatus with at least one ophthalmological laser and at least one control device, to a computer program and to a computer-readable medium.

### BACKGROUND

Treatment apparatuses and methods for controlling lasers for correcting an optical visual disorder of a cornea are known in the prior art. Therein, a pulsed laser and a beam focusing device can for example be formed such that laser beam pulses effect a photodisruption or an optical breakthrough in a focus situated within the tissue of the cornea, to separate a lenticule from the cornea for correcting the cornea. In the treatment with a treatment apparatus, for example for separating a lenticule, the eye is usually fixed by one or more contact elements of the treatment apparatus. Herein, the contact element can be a rigid element, for example a plano-concave lens, which is fitted onto the eye, in particular onto the cornea, in order that the eye is not moved in the treatment. Suction rings can also be used as the contact element, which fix the eye in a fixed position by means of negative pressure.

However, it is disadvantageous in such contact elements that a shape and thereby geometry of the cornea changes by the contact element and thus treatment coordinates, which have been determined in a non-deformed state of the cornea, can be erroneous, which deteriorates a treatment result.

The invention is based on the object to improve a treatment of an eye with a treatment apparatus.

### SUMMARY

This object is solved by the method according to the invention, the apparatuses according to the invention, the computer program according to the invention as well as the computer-readable medium according to the invention. Advantageous configurations with convenient developments of the invention are specified in the respective dependent claims, wherein advantageous configurations of the method are to be regarded as advantageous configurations of the treatment apparatus, of the control device, of the computer program and of the computer-readable medium and vice versa.

A first aspect of the invention relates to a method for adapting treatment coordinates for a treatment of an eye with an ophthalmological laser of a treatment apparatus, wherein the treatment apparatus includes a contact element for fixing the eye. As steps, the method includes acquiring at least a first image of the eye before the eye is fixed by the contact element, determining treatment coordinates of the eye by means of the first image and determining orientation points of the eye and the position thereof in the first image. Furthermore, the method includes acquiring a second image of the eye after the eye has been fixed by the contact element, wherein the position of the respective orientation points is determined in the second image, determining a transformation matrix based on the respectively determined positions of related orientation points in the first and the second image, and adapting the treatment coordinates by the determined transformation matrix.

Preferably, the thus adapted treatment coordinates can be provided as control data of the laser or of the treatment apparatus in a subsequent step to control the laser by means of the control data for correcting a visual disorder.

In other words, at least a first and a second image of the eye can be performed in the method, in which the iris of the eye is preferably visible. Therein, the first image can be imaged before fixing by the contact element and thus in the non-deformed state. Based on this first image, the treatment coordinates can preferably also be determined, by which a visual disorder of the eye can be corrected. Therein, the treatment coordinates can for example include positions of laser pulses in the cornea.

The second image of the eye is preferably imaged when the eye has been fixed by the contact element, thus is in a deformed state. In the two images, orientation points can then be searched in the eye, which are characteristic, such as for example a structure of the iris of the eye. Thus, an orientation point or a landmark can be determined in the first image and the same orientation point is searched in the second image. By the deformation by the contact element, therein, a change of the orientation point can occur between the two images, which can be described based on a vector. For multiple orientation points or landmarks, which occur in pairs in the two images, the respective changes or shifts can preferably be determined, from which a transformation matrix can be determined. Therein, the transformation matrix, which can preferably be an affine matrix, can describe how the orientation points change by the deformation.

Finally, the treatment coordinates can be adapted with the aid of the transformation matrix such that an effect of the deformation by the contact element can be compensated for. In adapting the treatment coordinates, either planned undeformed treatment positions can be deformed into newly adapted treatment positions by means of the transformation matrix or the treatment positions in the fixed state of the eye are retransformed to an undeformed state of the eye, from which the diagnosis has been made, by the transformation matrix, such that the treatment apparatus knows for these laser points, which correction is to apply.

Herein, the contact element can fix the eye by compressing or flattening the eye and/or comprise a suction device, which can suck the eye. Therein, the second image can be performed in contact with the contact element either with or without suction.

By the invention, the advantage arises that deformation effects can be compensated for in simple manner, which improves a treatment with the treatment apparatus.

The invention also includes forms of configuration, by which additional advantages arise.

A form of configuration provides that an affine transformation is performed by the transformation matrix. This means that the transformation matrix is an affine matrix, by which a map between two affine spaces is described, in which collinearity, parallelism and partial ratios are conserved. Thus, rotation, mirroring, scaling, shear and shift of the treatment coordinates can be described by the transformation matrix, wherein the maps are bijective.

A further form of configuration provides that the first image is performed by an external diagnostic device or a imaging device of the treatment apparatus, and wherein the second image is performed by the imaging device of the treatment apparatus. Thus, the first image can preferably be performed by an external diagnostic device for determining a visual disorder of the eye, wherein external means that the diagnostic device does not belong to the treatment apparatus. Alternatively, the first image can also be imaged by a imaging device of the treatment apparatus, for example when the patient is lying below the laser, wherein the first image is then performed just before docking to the contact element. Preferably, this image can be performed while viewing a coaxial fixing target. Furthermore, the eye can preferably be located close to (below 50 mm) the contact element. Thus, the eye is undeformed in this first image. In this form of configuration, the second image is always performed by the imaging device of the treatment apparatus when the eye is docked to the contact element and thus deformed.

Preferably, it is provided that the first image is performed by the external diagnostic device and a third image of the eye is acquired by the imaging device of the treatment apparatus between the first and the second image, before the eye is fixed by the contact element, wherein the positions of the orientation points are determined in the third image, wherein a calibration transformation matrix is determined from the respective positions of related orientation points in the first and the third image, wherein the transformation matrix is adapted by means of the calibration transformation matrix. In other words, thus, three images can preferably be performed, the first image by the external diagnostic device and thereafter the third image lying below the laser, but before the eye is fixed by the contact element. The third image can be imaged by the imaging device of the treatment apparatus. From the first and the third image, a calibration transformation matrix can then be determined based on related orientation points in the eye, by which differences between the imaging device of the diagnostic device and of the imaging device of the treatment apparatus can in particular be determined. Finally, the second image can be acquired by the imaging device of the treatment apparatus after the third image and the transformation matrix can be determined by means of the related orientation points of the first and the second image. This transformation matrix can then be adapted based on the calibration transformation matrix, for example by a matrix product, to describe the transformation of the treatment coordinates between the first image and the second image. Alternatively, the calibration transformation matrix can be determined by means of the orientation points of the first and the third image and thereafter a further transformation matrix between the orientation points of the second and the third image. Overall, the transformation matrix thus results from a matrix product of the matrix from image 1 and 3 and the matrix from image 2 and 3. In other words, the third image thus serves as a bridge image, which serves for calibrating the different imaging devices.

A further form of configuration provides that the respective images are performed in the same spectral range, in particular in the infrared spectral range or in the visible spectral range. In other words, the respective images are preferably performed with a same illumination, to better recognize related orientation points. Thus, all of the images can be performed in the infrared spectral range or in the visible spectral range. The infrared spectral range can begin from wavelengths greater than 690 nm and the visible spectral range can be present approximately in a range between 380 nm and 680 nm. Alternatively, the images can also be performed with different illuminations and/or wavelengths, if related orientation points can be found in these images. By this form of configuration, the advantage arises that related orientation points can be better determined.

A further form of configuration provides that the treatment coordinates are determined from the first image by means of a pupil center and/or a limbal ring of the eye. In other words, treatment centering can be determined in the image based on the pupil center and/or the limbal ring, which is a dark ring around the iris of the eye, at which the sclera encounters the cornea, in images. These positions can preferably also serve as orientation points for determining the transformation matrix at the same time. This results in the advantage that suitable and retrievable positions can be used for determining the treatment coordinates.

A further form of configuration provides that the orientation points are determined from the respective image based on characteristics of the iris of the eye. The iris in the eye has a structure individual and characteristic for each patient, which is advantageously suitable to determine orientation points. These orientation points can then be determined in the respective images, wherein a change of the orientation points between these images then serves for calculating the transformation matrix. Hereby, the advantage arises that orientation points can be determined in improved manner.

In a further form of configuration, it is provided that recentering and/or a cyclotorsion correction and/or a deformation correction of the treatment coordinates are performed by the transformation matrix. In other words, the treatment coordinates are recentered and shifted, respectively, and/or rotated and/or distorted, in particular a shear or pincushion distortion. Thus, treatment positions in the eye can be adapted by means of the transformation matrix to compensate for the deformation.

A further aspect of the present invention relates to a control device, which is configured to perform the above described method. This means, the control device can be formed to control the imaging devices and to calculate the transformation matrix. The above listed advantages arise. The control device can for example be configured as a control chip, control unit or application program (“app”). Preferably, the control device can comprise a processor device and/or a data storage. An appliance or an appliance component for electronic data processing is understood by a processor device. For example, the processor device can comprise at least one microcontroller and/or at least one microprocessor. Preferably, a program code for performing the method according to the invention can be stored on the optional data storage. The program code can then be adapted, upon execution by the processor device, to cause the control device to perform one of the above described embodiments of the method according to the invention.

A further aspect of the present invention relates to a treatment apparatus with at least one eye surgical laser for the treatment of a human or animal eye, in particular by means of optical breakthroughs and/or ablation and/or laser-induced refractive index change (LIRIC), and at least one control device, which is formed to execute the steps of the method according to the first aspect of the invention.

In a further advantageous configuration of the treatment apparatus according to the invention, the laser can be suitable to emit laser pulses in a wavelength range between 300 nm and 1400 nm, preferably between 900 nm and 1200 nm, at a respective pulse duration between 1 fs and 1 ns, preferably between 10 fs and 10 ps, and a repetition frequency of greater than 10 kilohertz (kHz), preferably between 100 kHz and 100 megahertz (MHz). The use of such lasers in the method according to the invention additionally has the advantage that the irradiation of the cornea does not have to be effected in a wavelength range below 300 nm. This range is subsumed by the term “deep ultraviolet” in the laser technology. Thereby, it is advantageously avoided that an unintended damage to the cornea is effected by these very short-wavelength and high-energy beams. Photodisruptive and/or ablative lasers of the type used here usually input pulsed laser radiation with a pulse duration between 1 fs and 1 ns into the corneal tissue. Thereby, the power density of the respective laser pulse required for the optical breakthrough can be spatially narrowly limited such that a high incision accuracy is ensured in the generation of the interfaces. In particular, the range between 700 nm and 780 nm can also be selected as the wavelength range.

In further advantageous configurations of the treatment apparatus, the control device can comprise at least one storage device for at least temporary storage of at least one control dataset, wherein the control dataset or datasets include(s) control data for positioning and/or for focusing individual laser pulses in the cornea; and can comprise at least one beam device for beam guidance and/or beam shaping and/or beam deflection and/or beam focusing of a laser beam of the laser.

Further features and the advantages thereof can be taken from the descriptions of the first inventive aspect, wherein advantageous configurations of each inventive aspect are to be regarded as advantageous configurations of the respectively other inventive aspect.

A further aspect of the invention relates to a computer program including commands, which cause the control device to execute the method steps according to the first inventive aspect.

A further aspect of the invention relates to a computer-readable medium (storage medium), on which the above mentioned computer program and the commands thereof, respectively, are stored. For executing the computer program, a computer or a computer cluster can access the computer-readable medium and read out the content thereof. For example, the storage medium is formed as a data storage, in particular at least partially as a volatile or non-volatile data storage. A non-volatile data storage can be a flash memory and/or an SSD (solid state drive) and/or a hard disk. A volatile data storage can be a RAM (random access memory). The commands can for example be present as a source code of a programming language and/or as an assembler and/or as a binary code.

Control data for the laser can include a respective dataset for positioning and/or for focusing individual laser pulses in the cornea. Additionally or alternatively, a respective dataset for adjusting at least one beam device for beam guidance and/or beam shaping and/or beam deflection and/or beam focusing of a laser beam of the respective laser can be included in the control data.

The method can include at least one additional step, which is executed if and only if an application case or an application situation occurs, which has not been explicitly described here. The step can for example include the output of an error message and/or the output of a request for inputting a user feedback. Additionally or alternatively, it can be provided that a default setting and/or a predetermined initial state is adjusted.

### BRIEF DESCRIPTION OF THE FIGURES

In the following, additional features and advantages of the invention are described based on the figure(s) in the form of advantageous execution examples. The features or feature combinations of the execution examples described in the following can be present in any combination with each other and/or the features of the embodiments. This means, the features of the execution examples can supplement and/or replace the features of the embodiments and vice versa. Thus, configurations are also to be regarded as encompassed and disclosed by the invention, which are not explicitly shown in the figures or explained, but arise from and can be generated by separated feature combinations from the execution examples and/or embodiments. Thus, configurations are also to be regarded as disclosed, which do not comprise all of the features of an originally formulated claim or extend beyond or deviate from the feature combinations set forth in the relations of the claims.

FIG. 1 is a schematically illustrated treatment apparatus according to an exemplary embodiment.

FIG. 2 is a schematic method diagram according to an exemplary embodiment.

In the figures, identical or functionally identical elements are provided with the same reference characters.

### DETAILED DESCRIPTION

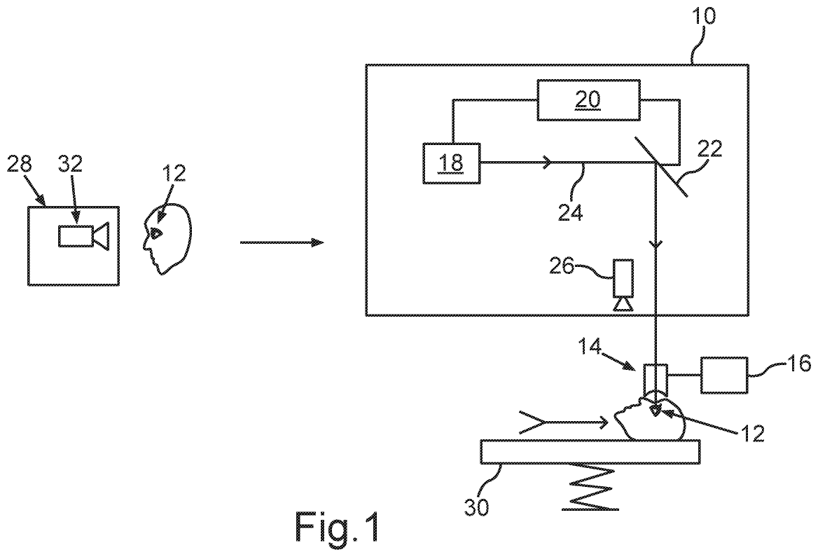

FIG. 1 shows a schematic representation of a treatment apparatus 10 with an ophthalmological laser 18 for the treatment of an eye 12, in particular of a cornea of the eye 12, by means of photodisruption and/or ablation and/or laser-induced refractive index change (LIRIC).

Besides the laser 18, the treatment apparatus 10 can comprise a control device 20, which can be formed to control the laser 18 by control data, such that it can emit pulsed laser pulses for example in a predefined pattern and at predetermined treatment coordinates, respectively, for treatment of the eye 12. Alternatively, the control device 20 can be a control device 20 external with respect to the treatment apparatus 10.

Furthermore, FIG. 1 shows that the laser beam 24 generated by the laser 18 can be deflected towards the eye 12 by means of a beam device 22, namely a beam deflection device, such as for example a rotation scanner. The beam deflection device 22 can also be controlled by the control device 20 to control the laser beam 24 to a preset position in the eye, in particular in the cornea of the eye 12.

Preferably, the laser 18 can be a photodisruptive and/or ablative laser, which is formed to emit laser pulses in a wavelength range between 300 nm and 1400 nm, preferably between 700 nm and 1200 nm, at a respective pulse duration between 1 fs and 1 ns, preferably between 10 fs and 10 ps, and a repetition frequency of greater than 10 kHz, preferably between 100 kHz and 100 MHz. In addition, the control device 20 optionally comprises a storage device (not illustrated) for at least temporary storage of at least one control dataset, wherein the control dataset or datasets include(s) control data for positioning and/or for focusing individual laser pulses in a cornea of the eye 12. The position data and/or focusing data of individual laser pulses can be generated based on predetermined control data, in particular from a previously measured topography and/or pachymetry and/or morphology.

Further, the treatment apparatus 10 can comprise a fixing device or a contact element 14, which is formed to fix the eye 12 to be treated in a position for the irradiation with the laser 18. For example, the contact element 14 can comprise a suction device 16, wherein the suction device 16 can be a vacuum pump, which generates a vacuum at a suction ring or ring segments capable of suction on a side of the contact element 14, which is oriented towards the eye 12. In other words, the suction ring can be fitted onto the eye 12 and the suction device 16, in particular the vacuum pump, can hold the eye 12 in position by generating a negative pressure.

In addition, the treatment apparatus 10 can include a imaging device 26, which can in particular acquire images of the eye 12, for example to center the eye for the laser 18 or to recognize previously determined treatment coordinates.

Furthermore, a diagnostic device 28 is illustrated, which includes at least one imaging device 32. Therein, the imaging device 32 can preferably comprise the same illumination as the imaging device 26 of the treatment apparatus 10. This means that the respective imaging devices 26 and 32 perform images in the same spectral range, in particular in the infrared spectral range and/or in the visible spectral range.

Thus, a first image of the eye 12 can be acquired by means of the imaging device 32 of the diagnostic device 28, based on which treatment coordinates for the treatment with the ophthalmological laser 18 of the treatment apparatus 10 can be determined. For determining the treatment coordinates, hereto, a pupil center and/or a limbal ring can preferably be used for centering the treatment coordinates. Furthermore, orientation points or landmarks can be set in the eye 12 from the first image, wherein they are preferably determined based on characteristics of the iris of the eye 12. Thus, a plurality of unique points in the eye 12 is preferably present, which can be retrieved in every image.

Thereafter, when the patient lies on the patient positioning device 30 in a treatment position below the treatment apparatus 10 and the eye 12 is fixed by the contact element 14, in particular by means of suction by the suction device 16, a second image of the eye 12 can be acquired by the imaging device 26 of the treatment apparatus 10. In this second image, the set orientation points can then be searched, which can be at least partially shifted by the fixing by the contact element 14.

From the positions of the related orientation points, which can be partially shifted between the first and the second image, a transformation matrix, in particular an affine transformation matrix, can then be determined, which describes a shift of the orientation points and thereby the deformation of the eye 12.

In order to be able to compensate for this deformation, the treatment coordinates, which have been determined by the diagnostic device 28, can be adapted based on the previously determined transformation matrix. This means that planned undeformed treatment positions can for example be calculated into new adapted treatment positions by means of the transformation matrix or the treatment positions in the fixed state of the eye 12 are retransformed to an undeformed state of the eye 12 by the transformation matrix, such that the treatment apparatus 10 knows for these treatment positions, which correction is to apply.

Particularly preferably, a third image can also be performed between the first image and the second image, wherein the third image is performed by the imaging device 26 of the treatment apparatus 10, preferably just before the patient is treated by the treatment apparatus 10, but the eye 12 has not yet been fixed by the contact element 14. Therein, the eye 12 can preferably be located near the contact element 14, for example in an area below 50 mm of distance. From the first image, which has been imaged by the imaging device 32 of the diagnostic device 28, and the third image in the non-fixed state, the respectively related orientation points can then be searched and possible deviations can be described by means of a calibration transformation matrix. Thus, the imaging device 32 of the diagnostic device 28 and the imaging device 26 of the treatment apparatus 10 can preferably be calibrated to each other. This calibration transformation matrix can then be used to adapt and thus to improve the transformation matrix.

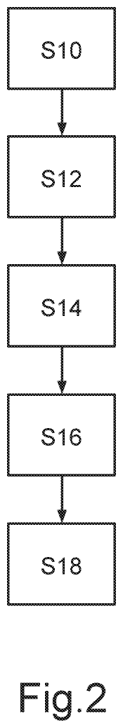

In FIG. 2, a schematic method diagram for adapting treatment coordinates for a treatment of an eye 12 with an ophthalmological laser 18 of a treatment apparatus 10 is illustrated. In a step S10, at least a first image of the eye 12 is imaged, before the eye is fixed by a contact element 14 of the treatment apparatus 10. Furthermore, treatment coordinates of the eye 12 are determined by means of the first image.

In a step S12, orientation points of the eye 12 and the position thereof are determined in the first image. Herein, the orientation points can be characteristics of an iris of the eye 12.

In a step S14, a second image of the eye 12 is imaged after the eye 12 has been fixed by the contact element 14, wherein the fixing can be fixed with or without suction by a suction device 16. The positions of the respective orientation points are then again determined from this second image.

In a step S16, a transformation matrix is determined in that the respective positions of related orientation points in the first and the second image are compared, wherein a deviation of the positions of the related orientation points describes the transformation by the transformation matrix.

Finally, the treatment coordinates can be adapted by the determined transformation matrix in a step S18, to thus adapt the irradiation positions by the laser 18 and to compensate for the deformation by the contact element 14. Then, the adapted treatment coordinates, which can be present as control data, can be used for controlling the laser 18.

In a further exemplary embodiment, it is provided that images of the eye 12 are imaged, for example by a diagnostic device 28. Alternatively, if the patient is at the treatment apparatus 10, at least one image of the eye 12 can be imaged, in particular with an infrared or visual illumination, while the eye 12 has not yet been fixed by the contact element 14, but is just about to, and while a coaxial fixing target is viewed. The pupil center can be detected from the images, in particular from the diagnostic image or the image below the laser or both, and the desired treatment centering can be determined in the images, for example depending on the pupil center or the limbal ring. In addition, peripheral landmarks, for example of the iris, can be detected from the images before fixing of the eye 12.

A further image, preferably with the same wavelength region in the infrared or visual spectral range, is imaged after the eye is docked to the contact element 14 and a suction device 16 fixes and retains the eye. From this image, the landmarks can then be searched and registered. An (affine) transformation matrix can be generated therefrom, which is generated from the detected positions of the landmarks in the images before and after fixing by the contact element 14. The treatment coordinates, which have been determined in the image before fixing, can thus be transformed to the image after fixing by the (affine) transformation matrix. Therein, an offset of the (affine) transformation matrix corresponds to a treatment centering, a rotational angle, which is calculated by the (affine) transformation matrix, can be used to correct a cyclotorsion axis and the transformation matrix can for example compensate for shears and pincushion distortions for the treatment positions.

In this manner, a compensation for the deformation can be achieved without performing theoretical/simulated models or statistical empirical corrections.

Overall, the examples show, how an affine corneal registration can be performed by the invention.

1. A method for adapting treatment coordinates for a treatment of an eye with an ophthalmological laser of a treatment apparatus, wherein the treatment apparatus includes a contact element for fixing the eye, wherein the method comprises:
acquiring at least a first image of the eye, before the eye is fixed by the contact element, and determining treatment coordinates of the eye by means of the first image;
determining orientation points of the eye and the position thereof in the first image;
acquiring a second image of the eye, after the eye has been fixed by the contact element, wherein the position of the respective orientation points is determined in the second image;
determining a transformation matrix based on the respectively determined positions of related orientation points in the first and the second image; and
adapting the treatment coordinates by the determined transformation matrix.

2. The method according to claim 1, wherein an affine transformation is performed by the transformation matrix.

3. The method according to claim 1, wherein the first image is performed by an external diagnostic device or a imaging device of the treatment apparatus and wherein the second image is performed by the imaging device of the treatment apparatus.

4. The method according to claim 3, wherein the first image is performed by the external diagnostic device and a third image of the eye is acquired by the imaging device of the treatment apparatus between the first and the second image, before the eye is fixed by the contact element, wherein the positions of the orientation points are determined in the third image, wherein a calibration transformation matrix is determined from the respective positions of related orientation points in the first and the third image, and wherein the transformation matrix is adapted by means of the calibration transformation matrix.

5. The method according to claim 1, wherein the respective images are performed in the same spectral range, in particular in an infrared spectral range or in a visible spectral range.

6. The method according to claim 1, wherein the treatment coordinates are determined from the first image by means of a pupil center and/or a limbal ring of the eye.

7. The method according to claim 1, wherein the orientation points are determined from the respective image based on characteristics of the iris of the eye.

8. The method according to claim 1, wherein recentering and/or a cyclotorsion correction and/or a deformation correction of the treatment coordinates are performed by the transformation matrix.

9. A control device, which is configured to perform a method according to claim 1.

10. A treatment apparatus with at least one ophthalmological laser for the treatment of a human or animal eye and at least one control device according to claim 9.

11. (canceled)

12. A non-transitory computer-readable medium on which a computer program is stored, the computer program including commands that cause at least one control device of a treatment apparatus with at least one ophthalmological laser for the treatment of a human or animal eye to execute a method according to claim 1.
